(19) United States
(12) Patent Application Publication
(10) Pub. No.: US 2005/0169944 A1
(43) Pub. Date: Aug. 4, 2005

(54) Increasing tumor oxygen content by administration of stressed cells

(76) Inventor: Thomas Ichim, Kitchener (CA)

Correspondence Address: KING & SPALDING LLP, 191 PEACHTREE STREET, N.E., 45TH FLOOR, ATLANTA, GA, 30303-1763, US

(21) Appl. No.: 10/508,308

(22) PCT Filed: Mar. 20, 2003
(86) PCT No.: PCT/CA03/00383

(30) Foreign Application Priority Data
Mar. 20, 2002 (CA) 2,377,442

(51) Int. Cl. A61K 39/00; A61K 33/00; A61N 1/30

(52) U.S. Cl. 424/277.1; 424/93.7; 424/600; 604/20

(57) ABSTRACT

The present invention provides a method of increasing oxygen content in tumors by administration of stressed cells, or inducing formation of stressed cells in vivo. Such treatment allows for increased efficacy of drugs, or radiation therapy, which normally would not be fully effective due to tumor hypoxia.

### FIELD OF THE INVENTION

This invention pertains to the field of medicine and in particular to the treatment of tumors.

### BACKGROUND OF THE INVENTION

Tumors are a rapidly expanding mass originating from one transformed cell. This rapid expansion does not allow for proper vascularization to occur. At the size of 2-3 mm³ the tumor would hypothetically stop growing because of its need for a blood supply to provide oxygen and nutrients (1). Unfortunately, the tumor can coerce the host endothelial cells to enter the growing mass and provide life support. This is accomplished, in part, by release of chemoattractant compounds from the growing tumor or from immune system cells which have entered the growing tumor. These compounds activate neighboring endothelial cells to migrate to the tumor, to cut through host tissue so that they can arrive at the tumor, and to start proliferating and forming new blood vessels for the tumor. This process is called angiogenesis (3).

Although the host-derived endothelium allows the tumor to grow, its vascular structure is much different from that of normal tissue. The tumor has no intratumor lymphatics, this does not permit fluid draining from tumour tissue and as a result high interstitial fluid pressure develops (2). The high interstitial pressure inhibits drugs from penetrating the whole tumor tissue, as well as forcing death of some tumor cells. This alteration of interstitial pressure combined with the rapid rate of tumor cell proliferation ends up forming a situation where the growing tumor is vascularized, but only to the limited extend that it needs for it's own survival. Tumor blood vessels do not contain smooth muscle lining, are resistant to control by the nervous system, and grow in a disorganized manner compared to vasculature in non-tumor tissue (3). An example of the difference between tumor and non-tumor vasculature is that the former relies on tumor secretion of vascular endothelial growth factor (VEGF) for its survival, whereas the later is insensitive to withdrawl of VEGF (4).

Tumors contain areas of hypoxia (4a). The cause of this is multifactorial and includes poor tumor perfusion by the blood (5), clotting of tumor blood vessels due to activated clotting factors on tumor endothelium (6), and the rapid rate of tumor growth. Hypoxia, and poor perfusion have been shown to negatively correlate with prognosis (7, 8). Cancer cells under hypoxic environments secrete matrix metalloproteases, which allow them to metastasize (9). In addition, hypoxia programs cancer cells and macrophages to secrete VEGF, a protein that stimulates angiogenesis as well as immune suppression (10). Hypoxia activates hypoxia inducible factor (HIF-1) a nuclear transcription factor which is important in promotion of angiogenesis (11).

Besides local hypoxia, late stage cancer patients have lower systemic hemoglobin levels compared to healthy controls, this is due in part to lower renal production of erythropoietin (11a). Lower hemoglobin implies less oxygen transport and therefore reduced tumor oxygenation. Studies aimed at increasing hemoglobin levels by administration of erythropoietin have shown increased efficacy of radiotherapy and chemotherapy (11b).

An important consideration of tumor hypoxia is the role it plays in protection of tumors from innate anti-tumor defense systems. For example tumor necrosis factor alpha (TNF-α) is a cytokine secreted by activated macrophages, which as the name implies has antitumor activity. Interestingly, the cytotoxicity of TNF-α to tumor cells is reduced under hypoxic conditions (12). Hypoxia also stimulates production of soluble TNF-α receptors which hypothetically may block systemic activities of this cytokine (13). Lymphokine activated killer (LAK) cells are effective in killing certain types of tumor cells in vitro and in vivo. The ability to generate these cells is depressed under conditions of hypoxia (14). Proliferation of lymphocytes in response to interleukin-2 is also inhibited during hypoxia (15).

Several therapeutic interventions such as radiation and certain types of chemotherapy are oxygen dependent. In 1953 the impact of tumor oxygenation on efficacy of radiation therapy was described by Gray et al (16). Since then a great number of studies confirming that tumor sensitivity to radiotherapy positively correlates with tumor oxygenation (reviewed in 17). Several other interventions such as etoposide, doxorubicin, camptothecin and vincristine therapy are oxygen dependent (17a).

Methods of increasing tumor oxygenation have been described. One such method involves exposing a patient to higher oxygen tension by use of a hyperbaric chamber. This approach has shown marginal increases in oxygenation of some tumors although clinical efficacy is a matter of debate. A randomized trial of squamous cell sarcoma patients treated with radiotherapy in the presence of air or hyperbaric oxygen demonstrated a significantly greater number of patients achieving clinical response in the hyperbaric oxygen group (18). Another randomized study assessing the ability of hyperbaric oxygen to increase efficacy of radiotherapy in cervical carcinoma patients demonstrated no beneficial effects (19). In addition to questionable clinical efficacy hyperbaric oxygen is a costly and sometimes dangerous procedure, which is not commonly used.

Inhalation of carbogen, a mixture of 95% oxygen and 5% carbon dioxide has also been shown to increase tumor oxygenation both in animal models (20,21) and in the clinical situation (22-24). An explanation for this effect is that carbon dioxide has vasodilatory functions in this setting which allows for better tumor perfusion of the high concentration of inhaled oxygen (25). Therapeutic benefits of carbogen therapy are mixed although some radiosensitizing effects have been observed alone (26-28), or in combination with nicotinamide in Phase I/H trials (29).

Due to the lack of established evidence of efficacy of the above methods used, it becomes apparent that novel methods of increasing tumor oxygen content are needed.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

The invention discloses a method of using agents which stress mammalian cells in such a manner so that administration of these stressed cells into a tumor bearing host will allow for increased oxygen content in the tumor. Another embodiment of the invention is stressing cells of a mammal in vivo through the administration of ozone gas. By increasing tumor oxygenation, therapeutic index of oxygen-dependent treatment modalities will be increased.

### BRIEF DESCRIPTION OF THE FIGURES

FIG. 1 illustrates administration of ozone stressed cells reduces tumor hypoxia as measured by Eppendorf™ probe.

FIG. 2 illustrates administration of ozone stressed cells enhances the ability of radiotherapy to decrease clonogenicity of tumors.

FIG. 3 illustrates administration of ozone stressed cells enhances ability of radiotherapy to inhibit tumor growth.

FIG. 4 illustrates administration of ozone stressed cells enhances ability of melphalan, an alkylating agent, to inhibit tumor growth.

FIG. 5 illustrates that administration of UVA+8-methoxypsoralen stressed cells increases tumor sensitivity to radiotherapy.

FIG. 6 illustrates that administration of UVA+8-methoxypsoralen stressed cells increases tumor sensitivity to melphalan, an alkylating agent.

FIG. 7 illustrates stressed mononuclear cells inhibits proliferation of endothelial cells.

FIG. 8 illustrates intravenous administration of ozone enhances tumor inhibitory effects of melphalan, an alkylating agent.

### DETAILED DESCRIPTION OF THE INVENTION

The invention provides a method for overcoming hypoxia in tumors through administration of a cell mixture or whole blood, which has been subjected ex vivo to biological stress and/or oxidative agents. These cells, chemical intermediates produced by the cells, or oxygen carried by these cells, induce biological cascades, which result in reduction of tumor hypoxia. The present invention provides various agents that can be used to stimulate this biological cascade. Increasing oxygen content of tumors renders them susceptible to various interventions including chemotherapy, radiotherapy and immune therapy.

Patient cells drawn from peripheral blood, or whole blood itself, is subjected to the stressful conditions. Alternatively, distinct subtypes of blood cells may be purified and subjected to stressful conditions. These conditions include but are not limited to alterations in temperature, alterations in osmotic balance, irradiation of the cells, serum depravation, treatment with gamma or ultraviolet radiation, treatment with ozone gas, or treatment with other agents which induce oxidative stress such as, but not limited to hydrogen peroxide. The stressed cells, intermediates released from the cells, or the mixture of the oxidative agents with cell-free portion of the blood are reintroduced to the patient either intravenously, subcutaneously, intramuscularly, or by other means of introducing agents into patients. The invention teaches that this treatment of purified cells or whole blood to stressors, will induce biological cascades which result in reduction of tumor hypoxia. Alternatively, injection of ozone gas at appropriate concentration in medical grade oxygen can result in stressing of cells in vivo, also causing biological cascades which result in the inhibition of tumor hypoxia. Such reduction of hypoxia is useful as an adjuvant to various cancer treatments known in the art or can be used alone to inhibit tumor growth by increasing oxygenation of tumors.

A method is provided for increasing oxygen content in tumor cells by cells which have been subjected to stress either outside of the body and subsequently reintroduced into the body, or by cells which have been stressed inside the body by administration of a stressing agent. As used herein, the term “stress” refers to conditions which alter the normal functions of cells. More specifically, several such conditions are described, without limiting the invention to specifics, these conditions include: exposure to oxidative agents such as ozone, treatment of cells with unphysiological conditions such as alterations in temperature, exposure of cells to radiation, or utilization of a chemosensitizer together with radiation such as the combination of 8-methoxypsoralen with ultraviolet A irradiation. Effects of stress on cells include alterations of normal functions, and in some cases apoptosis. The amount of stress administered to a cell to achieve the desired effect of a cell population capable of increasing oxygen content of tumors once the cells are introduced into the tumor bearing animal, is decided by the tumor type, type of cell stressed, and individual characteristics of the patient. The practitioner of the invention disclosed will possess access to techniques that allow for individual determination of the most optimum method of practicing the invention. For example, quantification of tumor oxygenation can be performed using Eppendorf™ probes in order to modify the types and amounts of the indicated components of this invention to achieve maximal oxygenation of the tumor(s).

In one embodiment, whole blood is taken from the patient in a desired volume, preferentially but not limited to a volume of 10 ml and ozone gas is bubbled through it in a sterile container. Cells can be stressed by bubbling 5-20 μg of ozone per ml of oxygen at a flow rate of 220 ml/minute for 2 minutes in a sterile glass flask. Ozone gas may also be mixed with the blood using methods known in the art, including those described by Wainwright (30), Muller (31), or by Williams (32) The concentration of ozone gas being bubbled through the blood, as well as, the amount of time that it is bubbled through, is determined on the appropriate amount of stressed cells needed to achieve a decrease in tumor hypoxia. Stressing of blood cells can be measured by surrogate markers such as secretion of interleukin 10 using ELISA, release of heat shock proteins, or amount of apoptotic lymphocytes using AnnexinV-FITC staining. The treated blood is then administered in the patient intramuscularly or subcutaneously. Such treatment can be performed on a schedule depending on the needs of the patient. A typical treatment protocol is administration of the stressed blood cells three times a week for several months. This embodiment can be practiced by one skilled in the art with various modifications while not departing from the spirit of the invention, that is, the administration of the stressed cells in order to decrease tumor hypoxia.

Another embodiment of the invention involves the extracting 50 to 100 ml of the patient's blood, ozonating it ex vivo, using an appropriate concentration of ozone and an appropriate time span to induce formation of stressed cells, and readministering it to the same patient. Following ozonation, the stressed blood cells may be readministered in the form of a normal drip-infusion. Blood cells can be stressed by bubbling 5-20 μg of ozone per ml of oxygen at a flow rate of 220 ml/minute for 2 minutes in a sterile glass flask. The rate of reinfusion is preferable, but not limited to 90 drips per minute.

Yet another embodiment of the invention disclosed involves ozonation of blood through a extra-corporeal loop apparatus where a tube is placed in the artery of the patient, the blood flows through an ozone generator, the blood is mixed with the ozone ex vivo, and returned to the patient via a tube placed in a vein. Such an apparatus has been previously described in the art hich has been described in the art (30).

In the above embodiments care must be taken not to administer ozone at a high enough concentration or for a long enough duration to cause hemolysis. Ozone is usually administered to the blood as a mixture with pure oxygen but other methods of administration may be devised by one skilled in the art without departing from the spirit of the disclosed invention. A concentration of ozone used by our group, which was found optimal was 5-70 μg/ml, although 30-300 μg/ml may also be used.

An embodiment of the invention involves administration of apoptotic cells to the patient. Purification of lymphocytes may easily be performed by centrifugation of whole blood on a density gradient. Cells from the patient may then be purified of erythrocytes and treated by various means such that apoptosis is induced. Such means are commonly known in the art, examples of which include administration of protein synthesis inhibitors or nucleoside analogues, physical changes in osmotic pressure, ligation of death receptors, or treatment with radiation. Using the system described for treatment of psoriasis, cells may be sensitized with a chemical and then exposed to UV irradiation as described by McLaughlin et al (33). These cells can then be purified for apoptotic bodies, or used as an unpurified inoculum. Administration of these cells may take place via subcutaneous, intramuscular, or intravenous route depending on the condition of the patient and the effect desired.

The work of Bolton (34) described a novel means of inducing systemic antiinflammatory biological responses by administration of cells stressed with heat and/or UV radiation, while being exposed to ozone. An embodiment of the invention described herein is to use the method of Bolton for reduction of tumor hypoxia.

Another embodiment involves stressing of distinct cell populations found in circulation. Purified cell populations from whole blood can have potent effects at decreasing tumor hypoxia An embodiment of this invention involves purification of a cell population, stressing of the cell, and readministration in order to reduce tumor hypoxia. These cells may also be grown, expanded, and/or differentiated ex vivo before administration of the stressor. A preferred embodiment involves purification of circulating dendritic cells from the patient according to methods such as Magnetic Activated Cell Sorting (MACS) or flow sorting, stressing the dendritic cells with ozone, or any of the stressors mentioned above, and readministration of the cells to the patient. In the cases where substantial numbers of dendritic cells can not be purified, patient monocytes can be induced to differentiate into dendritic cells by treatment with granulocyte-macrophage colony stimulating factor in conjunction with interleukin-4 as previously described (35). Another purified population of cells may be T cells. Further purification based on phenotypic markers can be performed according to the amount and type of response desired in order to reduce tumor hypoxia.

Another embodiment involves stressing of cells through subjecting them to conditions which are unphysiological. The stress signaling pathways seem to converge to common intracellular mechanisms and nuclear transcription factors. For this reason cells which are subjected to serum starvation turn on the same, or very similar apoptotic pathways as cells which are “stressed” by radiation or hyperthermia. The cancer anti-hypoxic utility of administration of cells stressed by unphysiological conditions is an embodiment of the invention described herein.

Another embodiment involves administration of an ozone/oxygen mixture directly into circulation of a mammal through intravenous injection. This procedure increases tumor oxygenation by inducing formation of stressed cell in vivo.

### EXAMPLES

### Example I

Reduction of Hypoxia in the KHT Fibrosarcoma Model by Ozone Stressed Blood

10 ml of blood was pooled from 10 C3H mice collected by cardiac puncture in sodium citrate tubes. Cells were stressed by bubbling 5 or 20 μg of ozone per ml of oxygen at a flow rate of 220 ml/minute for 2 minutes in a sterile glass flask. As a control, blood was placed in the glass flask for 2 minutes and medical grade oxygen was bubbled through it at the same flow rate.

For evaluating the effect of stressed cells on tumor oxygenation, 9-12 week old male C3H mice were divided into groups of 10, all of which were inoculated with 2×10⁵ KHT sarcoma cells into the left gastrocnemius muscle. Treatments were performed for 5 consecutive days starting two days after tumor cell inoculation.

Group I was given no treatment. Group II was treated with a 50 μl intramuscular injection of untreated blood. Group III was treated with a 50 μl intramuscular injection of blood treated with ozone at 5 μg/ml (described above). Group IV was treated with a 50 μl intramuscular injection of blood treated with ozone at 20 μg/ml (described above).

On day 7 after tumor inoculation, an Eppendorf computerized fine-needle polarographic electrode probe (Eppendorf, Hamburg, Germany) was used for intratumor measurement of hypoxia. In order to assess hypoxic areas, the electrode was inserted 1 mm into the tumor. The probe was moved through the tumor in increments of 0.8 mm followed by withdrawing the probe 0.3 mm before taking the measurement. 1.4 second intervals were allowed between re-measurements. An average of 5 parallel insertions were performed in each tumor with an average of 50 measurements taken per tumor. Oxygenation is reported as mean pO2 (mmHg).

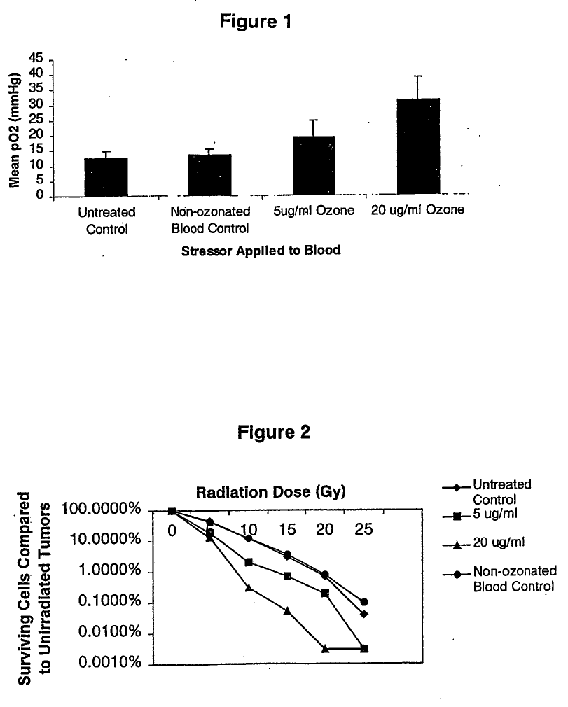

As illustrated in FIG. 1, administration of ozonated blood from syngeneic mice for 5 consecutive days resulted in increased tumor oxygenation compared to mice treated with blood that was not ozonated. Increased oxygen content in tumors was higher in animals which received blood that was treated with 20 μg/ml compared to those treated with 5 μg/ml.

### Example II

Reduction of Tumor Clonogenicity in the KHT Fibrosarcoma Model by Ozone Stressed Blood

9-12 week old male C3H mice were divided into groups of 10, all of which were inoculated with 2×10⁵ KHT sarcoma cells into the left gastrocnemius muscle. Treatments were performed for 5 consecutive days starting two days after tumor cell inoculation.

Group I was given no treatment. Group II was treated with a 50 μl intramuscular injection of untreated blood. Group III was treated with a 50 μl intramuscular injection of blood treated with ozone at 5 μg/ml (described above). Group IV was treated with a 50 μl intramuscular injection of blood treated with ozone at 20 μg/ml (described above).

One day 7 post-tumor inoculation, tumors from mice in all groups were irradiated with a ¹³⁷CS source at 3 Gy/min for varying time points to administer doses of 0-25 Gy. Tumors were extracted from mice and made into single cell suspensions by mechanical disassociation, and treatment with trypsin and DNase I. Single cells were washed with phosphate buffered saline and plated at 5×10⁵ cells/ml in a 24 well plate. As feeder cells 10⁴ lethally irradiated KHT cells/well were plated. The culture media was 0.2% agar in α-MEM media with 10% fetal calf serum. Cells were incubated for 14 days at 37 Celsius, with 5% CO₂ in a fully humidified atmosphere. Colonies with greater than 50 cells were enumerated.

As illustrated in FIG. 2, the higher concentration of ozone which was applied to the blood cells, the more sensitive the tumor cells became to radiotherapy. In contrast, administration of unstressed mouse blood did not affect radiosensitivity.

### Example III

Enhanced Inhibition of Tumor Growth by Ozone Stressed Cells

C3H mice were inoculated with KHT tumors as described above. At about day 7-8 when tumors reached 8 mm in circumference mice were divided into 4 groups of 10 mice per group. Group I was the untreated control, Group II was administered 15 cGy, Group III was administered 15cGy together with an intramuscular injection of 50 μl of untreated syngeneic blood. Group IV was injected with 50 μl of syngeneic blood treated with 20 μl ozone/ml of oxygen as described above. Injections of untreated, and treated blood were administered one hour before radiotherapy, and for 4 subsequent days. Tumor size was evaluated daily and the time until tumors reached 16 mm was recorded.

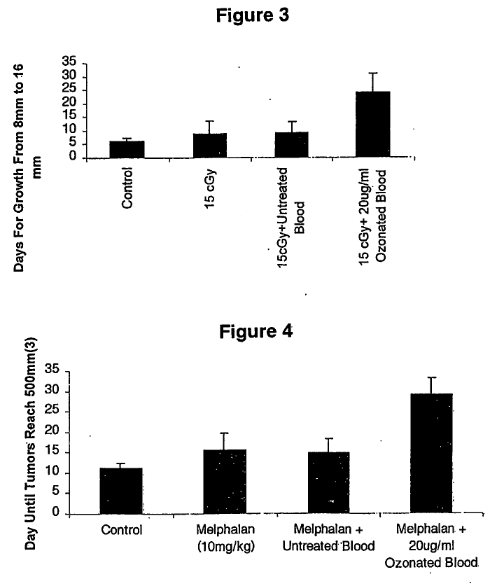

As illustrated in FIG. 3, treatment with ozone-stressed blood cells resulted in a potent prolongation of time it took for tumor growth to occur.

### Example IV

Administration of Ozone Stressed Cells Increases Sensitivity of Tumors to Alkylating Agents

8 week old male C3H mice were inoculated with 2×10⁶ FSaIIC murine fibrosarcoma cells in the hind limb. When tumors reached a volume of 50 mm³, animal were divided into 4 groups of 10 mice. Group I was untreated, Group II was administered a dose of 10 mg/kg melphalan by intraperitoneal injection. Group III was administered melphalan together with 50 μl of untreated syngeneic blood injected intramuscularly in the non-tumor bearing hind limb. Group IV was administered melphalan with 50 μl of syngeneic blood treated with 20 μl ozone/ml of oxygen. Following the initial melphalan injection, animals in Groups III and IV continued receiving intramuscular injections of 50 μl of untreated syngeneic blood, or 50 μl of syngeneic blood treated with 20 μl ozone/ml of oxygen, respectively for 4 days, one injection per day. All animals were observed until tumors attained a volume of 500 mm³. The delay in tumor time to achieve 500 mm³ volume is illustrated in FIG. 4.

### Example V

Administration of Cells Stressed By UVA Together with 8-Methoxypsoralen Increases Sensitivity of Tumor Cells To Radiotherapy

C3H spleens were extracted from male mice and made into a single cell suspension by mechanical dissociation. Erythrocytes were lysed using hypotonic solution, and mononuclear cells were washed in phosphate buffered saline. These mononuclear cells were subsequently incubated, at 37 Celsius for 20 minutes with 200 ng/ml of 8-methoxypsoralen in complete RPMI media at a concentration of 10⁶ cells per ml. Cells were subsequently exposed to a 2 J/cm² dose of UVA (peak of emission: 365 nm). Exposed cells were allowed to incubate for 18 hours, at 37 Celsius, followed by washing in phosphate buffered saline and subcutaneous injection into the non-tumor bearing hind limb of the C3H mice.

C3H mice were inoculated with KHT tumors as described above. At about day 7-8 when tumors reached 8 mm in circumference mice were divided into 4 groups of 10 mice per group. Group I was the untreated control, Group II was administered 15 cGy, Group III was administered 15cGy together with an intramuscular injection of 1×10⁶ syngeneic untreated mononuclear cells. Group IV was injected with 1×10⁶ syngeneic mononuclear cells that were stressed using UVA and 8-methoxypsoralen as described above. Injections of untreated, and treated mononuclear cells were administered one hour before radiotherapy, and for 4 subsequent days, once per day. Tumor size was evaluated daily and the time until tumors reached 16 mm was recorded.

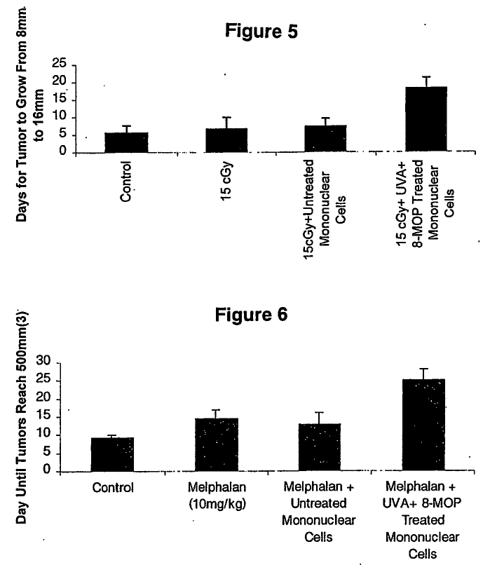

As illustrated in FIG. 5, treatment with stressed lymphocytes resulted in a potent prolongation of time it took for tumor growth to occur.

### Example VI

Administration of Cells Stressed By UVA Together with 8-Methoxypsoralen Increases Sensitivity of Tumor Cells To Alkylating Agents

8 week old male C3H mice were inoculated with 2×10⁶ FSaIIC murine fibrosarcoma cells in the hind limb. When tumors reached a volume of 50 mm³, animal were divided into 4 groups of 10 mice. Group I was untreated, Group II was administered a dose of 10 mg/kg melphalan by intraperitoneal injection. Group HI was administered melphalan together with an intramuscular injection of 1×10⁶ syngeneic untreated mononuclear cells. Group IV was administered melphalan with 1×10⁶ syngeneic mononuclear cells that were stressed using UVA and 8-methoxypsoralen as described above. Following the initial melphalan injection, animals in Groups III and IV continued receiving intramuscular injections of 1×10⁶ syngeneic untreated mononuclear cells, or 1×10⁶ syngeneic mononuclear cells that were stressed using UVA and 8-methoxypsoralen, respectively for 4 days, one injection per day. All animals were observed until tumors attained a volume of 500 mm³. The delay in tumor time to achieve 500 m³ volume is illustrated in FIG. 6.

### Example VII

Stressed Mononuclear Cells Inhibits Proliferation of Endothelial Cells

Murine mononuclear cells were stressed with 5 μg/ml or 20 μg/ml of ozone, or stressed by treatment with 8-methoxypsoralen and UVA, as described in the previous examples. 5×10⁴ stressed, or untreated mononuclear cells were added to confluent human umbilical vein endothelial cells (HUVEC) in 96 well plates. Cells were cultured in EBM-2™ media. Co-culture of stressed cells with HUVEC was performed for 24 hours. Proliferation of HUVEC was assessed by pulsing the 96 well plate with 1 μCi tritiated thymidine per well for 18 hours. Cells were then harvested on a Wallac™ harvester, and radioactivity was measured using scintillation counting.

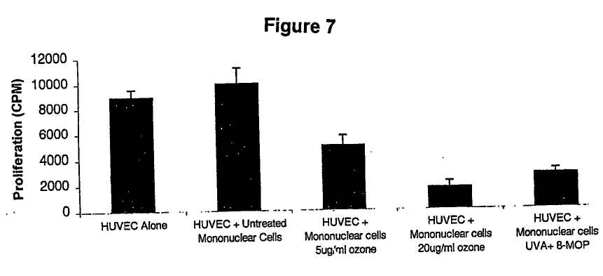

As seen in FIG. 7, mononuclear cell had minimal activity on proliferation of HUVEC cells. In contrast cells stressed by ozone and UVA+8-methoxypsoralen had an antiproliferative effect.

### Example VIII

In Vivo Stressing of Cells Increases Sensitivity of Tumors to Alkylating Agents

In order to stress cells in vivo, ozone at 5 μg per ml of oxygen, or 20 μg per ml of oxygen was injected directly into the tail vein of tumor bearing mice. Such injections are performed by filling a 1 cc syringe with the indicated concentration of ozone in medical grade oxygen. The gas is slowly injected into the tail vein so not to induce embolism. Due to the high solubility of oxygen and ozone in blood, embolism formation is not usually a problem.

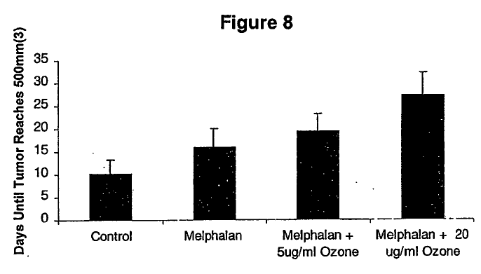

8 week old male C3H mice were inoculated with 2×10⁶ FSaIIC murine fibrosarcoma cells in the hind limb. When tumors reached a volume of 50 mm³, animal were divided into 4 groups of 10 mice. Group I was untreated, Group II was administered a dose of 10 mg/kg melphalan by intraperitoneal injection. Group III was administered melphalan together with 50 μl of 5 μg of ozone per ml of oxygen by tail vein injection. Group IV was administered melphalan with 50 μl of 20 μg of ozone per ml of oxygen by tail vein injection. Following the initial melphalan injection, animals in Groups III and IV continued receiving intravenous injections of 50 μl of 5 μg of ozone per ml of oxygen, or 50 μl of 20 μg of ozone per ml of oxygen, respectively for 4 days, one injection per day. All animals were observed until tumors attained a volume of 500 mm³. The delay in tumor time to achieve 500 m³ volume is illustrated in FIG. 8. Treatment with intravenous ozone was well tolerated and both concentrations increased the tumor inhibitory effects of melphalan.

The invention discloses several biotherapeutic utilities of DU-145 supernatant. It is to be understood that the above examples are presented only for clarity and that the invention may take other embodiments as practiced by one skilled in the art. Through routine experimentation, one skilled in the arts will recognize many equivalents to the specific materials and procedures described herein. These equivalents are intended to be encompassed with the scope of the claims of the disclosed invention.

The invention being thus described, it will be obvious that the same may be varied in many ways. Such variations ar not to be regarded as a departure from the spirit and scope of the invention, and all such modifications as would be obvious to one skilled in the art are intended to be included within the scope of the following claims.

### REFERENCES

1. Folkman J. Tumor angiogenesis. Adv Cancer Res. 1985; 43: 175-203
2. Jain R K. Haemodynamic and transport barriers to the treatment of solid tumours. Int J Radiat Biol. 1991 July-August; 60(1-2): 85-100
3. Tumour angiogenesis/edited by Roy Bicknell, Claire E. Lewis, and Napoleone Ferrara. Oxford, [England]; New York: Oxford University Press, 1997.
4. Benjamin L E, Golijanin D, Itin A, Pode D, Keshet E. Selective ablation of immature blood vessels in established human tumors follows vascular endothelial growth factor withdrawal. J Clin Invest. 1999 January; 103(2): 159-65.
4a. Williams K J, Cowen R L, Stratford I J. Hypoxia and oxidative stress. Tumour hypoxia—therapeutic considerations. Breast Cancer Res. 2001; 3(5): 328-31
5. Fenton B M, Lord E M, Paoni S F. Intravascular HBO(2) saturations, perfusion and hypoxia in spontaneous and transplanted tumor models. Int J Cancer. 2001 Sep. 1; 93(5): 693-8.
6. Gouin-Thibault I, Achkar A, Samama M M. The thrombophilic state in cancer patients. Acta Haematol. 2001; 106(1-2): 33-42
7. Dunst J, Ahrens S, Paulussen M, Burdach S, Jurgens H. Prognostic impact of tumor perfusion in MR-imaging studies in Ewing tumors. Strahlenther Onkol. 2001 March; 177(3): 153-9.
8. Rudat V, Stadler P, Becker A, Vanselow B, Dietz A, Wannenmacher M, Molls M, Dunst J, Feldmann H J. Predictive value of the tumor oxygenation by means of pO2 histography in patients with advanced head and neck cancer. Strahlenther Onkol. 2001 September;177(9):462-8.
9. Canning M T, Postovit L M, Clarke S H, Graham C H. Oxygen-mediated regulation of gelatinase and tissue inhibitor of metalloproteinases-1 expression by invasive cells. Exp Cell Res. 2001 Jul. 1;267(1):88-94.
10. Semenza G L. HIF-1: using two hands to flip the angiogenic switch. Cancer Metastasis Rev. 2000; 19(1-2): 59-65
11. Pugh C W, Gleadle J, Maxwell P H. Hypoxia and oxidative stress in breast cancer. Hypoxia signalling pathways. Breast Cancer Res. 2001; 3(5): 313-7.
11a. Kurzrock R. The role of cytokines in cancer-related fatigue. Cancer. 2001 September 15; 92(6 Suppl): 1684-8.
11b. Littlewood T J. The impact of hemoglobin levels on treatment outcomes in patients with cancer. Semin Oncol. 2001 April; 28(2 Suppl 8): 49-53.
12. Naldini A, Cesari S, Bocci V. Effects of hypoxia on the cytotoxicity mediated by tumor necrosis factor-alpha. Lymphokine Cytokine Res. 1994 August; 13(4): 233-7.
13. Scannell G, Waxman K, Kaml G J, Ioli G, Gatanaga T, Yamamoto R, Granger G A. Hypoxia induces a human macrophage cell line to release tumor necrosis factor-alpha and its soluble receptors in vitro. J Surg Res. 1993 April; 54(4): 281-5.
14. Ishizaka S, Kimoto M, Tsujii T. Defect in generation of LAK cell activity under oxygen-limited conditions. Immunol Lett. 1992 May; 32(3): 209-14.
15. Loeffler D A, Juneau P L, Masserant S. Influence of tumour physico-chemical conditions on interleukin-2-stimulated lymphocyte proliferation. Br J Cancer. 1992 October; 66(4): 619-22.
16. Gray L H, Conger A D, Ebert M, Hornsey S, Scott O C. The concentration of oxygen dissolved in tissues at the time of irradiation as a factor in radiotherapy. Br J Radiol 26: 638-48, 1953.
17. Hill R P. Cellular basis of radiotherapy. In: Tannock I F, Hill R P, editors. The basic science of oncology. 2^{nd} ed. New York: McGraw-Hill; pp 259-275, 1992.
17a. 17a). Tomida A, Tsuruo T. Drug resistance mediated by cellular stress response to the microenvironment of solid tumors. Anticancer Drug Des. 1999 April; 14(2): 169-77.
18. Haffty B G, Hurley R, Peters L J. Radiation therapy with hyperbaric oxygen at 4 atmospheres pressure in the management of squamous cell carcinoma of the head and neck: results of a randomized clinical trial. Cancer J Sci Am. 1999 November-December; 5(6): 341-7.
19. Dische S, Saunders M I, Sealy R, Werner I D, Verma N, Foy C, Bentzen S M. Carcinoma of the cervix and the use of hyperbaric oxygen with radiotherapy: a report of a randomised controlled trial. Radiother Oncol. 1999 November; 53(2): 93-8.
20. Hartmann K A, van der Kleij A J, Carl U M, Hulshof M C, Willers R, Sminia P. Effects of hyperbaric oxygen and normobaric carbogen on the radiation response of the rat rhabdomyosarcoma R1H(1). Int J Radiat Oncol Biol Phys. 2001 November. 15; 51(4): 1037-44.
21. Horsman M R, Khalil A A, Nordsmark M, Grau C, Overgaard J. Relationship between radiobiological hypoxia and direct estimates of tumour oxygenation in a mouse tumour model. Radiother Oncol. 1993 July; 28(1): 69-71.
22. Powell M E, Collingridge D R, Saunders M I, Hoskin P J, Hill S A, Chaplin D J. Improvement in human tumour oxygenation with carbogen of varying carbon dioxide concentrations. Radiother Oncol. 1999 February; 50(2): 167-71.
23. Aquino-Parsons C, Green A, Minchinton A I. Oxygen tension in primary gynaecological tumours: the influence of carbon dioxide concentration. Radiother Oncol. 2000 October; 57(1): 45-51.
24. Partridge S E, Aquino-Parsons C, Luo C, Green A, Olive P L. A pilot study comparing intratumoral oxygenation using the comet assay following 2.5% and 5% carbogen and 100% oxygen. Int J Radiat Oncol Biol Phys. 2001 Feb. 1;49(2): 575-80.
25. Griffiths J R, McIntyre D J, Howe F A, McSheehy P M, Ojugo A S E, Rodrigues L M, Wadsworth P, Price N M, Lofts F, Nicholson G, Smid K, Noordhuis P, Peters G J, Stubbs M. Issues of normal tissue toxicity in patient and animal studies—effect of carbogen breathing in rats after 5-fluorouracil treatment. Acta Oncol. 2001; 40(5): 609-14.
26. Martinez J C, Villar A, Cabezon M A, de Serdio J L, Fuentes C, Espineira M, Perez M D, Gil J, Artazkoz J J, Borque C, Suner M, Saavedra J A. Hyperfractionated chemoradiation with carbogen breathing, with or without erythropoietin: a stepwise developed treatment schedule for advanced head-and-neck cancer. Int J Radiat Oncol Biol Phys. 2001 May 1;50(1): 47-53.
27. Marcial V A, Pajak T F, Kramer S, Davis L W, Stetz J, Laramore G E, Jacobs J R, Al-Sarraf M, Brady L W. Radiation Therapy Oncology Group (RTOG) studies in head and neck cancer. Semin Oncol. 1988 February; 15(1): 39-60.
28. Dische S, Rojas A, Rugg T, Hong A, Michael B D. Carbogen breathing: a system for use in man. Br J Radiol. 1992 January; 65(769): 87-90.
29. Hoskin P J, Saunders M I, Dische S. Hypoxic radiosensitizers in radical radiotherapy for patients with bladder carcinoma: hyperbaric oxygen, misonidazole, and accelerated radiotherapy, carbogen, and nicotinamide. Cancer. 1999 Oct. 1; 86(7): 1322-8.
30. U.S. Pat. No. 6,027,688 Wainwright
31. U.S. Pat. No. 4,968,483 Muller
32. U.S. Pat. No. 5,612,226 Williams
33. U.S. Pat. No. 5,984,887 McLaughlin
34. U.S. Pat. No. 6,204,058 Bolton
35. U.S. Pat. No. 5,849,589 Tedder

1. A method for increasing oxygen content in tumors, the method comprising the steps of:
a) withdrawing cells from a mammal;
b) creating stressed cells by subjecting said cells to a stressor; and
c) re-administering the stressed cells to the mammal.

2. The method of claim 1 wherein the said cells used comprise whole blood, purified populations of blood cells, or mixtures of host cells at a specific ratio.

3. The method of claim 2 wherein purified T cells are used.

4. The method of claim 2 wherein purified dendritic cells are used.

5. The method of claim 1 wherein the stressor comprises of heat, UV irradiation, gamma irradiation, or treatment with ozone gas.

6. The method of claim 1 wherein the stressor comprises of placing the cells in an environment distinct from which they normally reside.

7. The method of claim 5 wherein the stressors described are used alone or in combination.

8. The method of claim 5 wherein stress by heat is accomplished through elevation of the temperature above 37° C. but below 55° C.

9. The method of claim 5 wherein stress by UV irradiation consists of exposing cells to UV light of wavelength 400 nm or shorter.

10. The method of claim 5 wherein stress by gamma irradiation is accomplished by exposure to a ¹³⁷Cs gamma source in the appropriate time length to administer 4-50 Gy of radiation.

11. The method of claim 5 wherein stress by ozone treatment is achieved by mixing cells in combination of ozone in oxygen at concentrations of ozone between 0.1 μg/ml to 100 μg/ml.

12. The method of claim 5 where stress may be applied by the use of a sensitizing agent, which increases susceptibility to the stressor.

13. The method of claim 1 where stressed cells may be generated in vivo by introduction of ozone into systemic circulation.

14. The method of claim 12 wherein the sensitizer is a psoralen or the psoralen derivative, 8-methoxypsoralen.

15. The method of claim 12 wherein 8-methoxypsoralen.

16. A method for inhibiting proliferation of tumor endothelial cell by increasing oxygen content in the microenvironment.

17. A method of inhibiting proliferation of endothelial cells by contact with stressed cells.

18. A method of increasing efficacy of cancer therapies whose efficacy is dependent on oxygen content of the tumor.

19. The method of claim 18 where the said therapies are radiotherapies, chemotherapies or immunotherapies.

20. The method of claim 18 where chemotherapies consist of at least one of the following: an alkylating agent, lytic agent, DNA intercalator, enzyme inhibitor, or antimetabolite.

21. The method of claim 18, where chemotherapies consist of at leas one of the following: vinblastin, streptozotocin, thiotepa, carmustine, busulfan, melphalan, chlorambucil, cisplatin, hydroxyurea, prednisone, actinomycin D, methotrexate or doxorubicin hydrochloride.
